(19) United States
(12) Patent Application Publication
(10) Pub. No.: US 2007/0099283 A1
(43) Pub. Date: May 3, 2007

(54) Recombinant proteinase k

(76) Inventors: Rainer Mueller, Penzberg (DE); Johann-Peter Thalhofer, Weilheim (DE); Bernhard Rexer, Weilheim (DE); Rainer Schmuck, Benediktbeuern (DE); Frank Geipel, Penzberg (DE); Stephan Glaser, Seeshaupt (DE); Helmut Schoen, Penzberg (DE); Thomas Meier, Munich (DE); Rainer Rudolph, Halle (DE); Hauke Lilie, Halle (DE); Bjoern Schott, Giessen (DE)

Correspondence Address: Roche Diagnostics Corporation, 9115 Hague Road, PO Box 50457, Indianapolis, IN, 46250-0457, US

(21) Appl. No.: 10/467,532

(22) PCT Filed: Feb. 8, 2002
(86) PCT No.: PCT/EP02/01322

§ 371 Date: Apr. 5, 2004

(30) Foreign Application Priority Data
Feb. 9, 2001 (DE) 101 05 912.4

(51) Int. Cl. C12N 9/58

(52) U.S. Cl. 435/223

(57) ABSTRACT

The invention concerns recombinant proteinase K. Furthermore a method for producing recombinant proteinase K is disclosed, which is characterized in that a) a host cell is transformed with a recombinant nucleic acid which codes for the zymogenic precursor of proteinase K, b) the host cell is cultured in such a manner that the zymogenic precursor of proteinase K is formed in the form of inclusion bodies in the host cell, c) the inclusion bodies are isolated and natured under conditions which result in the formation of the protease part of the zymogenic precursor in its natural conformation, d) the natured proteinase K is activated and purified.

The present invention concerns the preparation of recombinant proteinase K from Tritirachium album Limber and corresponding methods for the expression, in vitro naturation and activation of the recombinant proteinase K.

Proteinase K (E.C. 3.4.21.64, also known as endopeptidase K) is an extracellular endopeptidase which is synthesized by the fungus Tritirachium album Limber. It is a member of the class of serine proteases with the typical catalytic triad Asp³⁹-His⁶⁹-Ser²²⁴ (Jany, K. D. et al. (1986) FEBS Letters Vol. 199(2), 139-144). Since the sequence of the polypeptide chain of 279 amino acids in length (Gunkel, F. A. and Gassen, H. G. (1989) Eur. J. Biochem. Vol. 179(1), 185-194) and the three dimensional structure (Betzel, C. et al. (1988) Eur. J. Biochem. Vol. 178(1), 155-71) has a high degree of homology to bacterial subtilisins, proteinase K is classified as a member of the subtilisin family (Pahler, A. et al. (1984) EMBO J. Vol. 3(6), 1311-1314; Jany, K. D. and Mayer, B. (1985), Biol. Chem. Hoppe-Seyler, Vol. 366(5), 485-492). Proteinase K was named on the basis of its ability to hydrolyse native keratin and thus allows the fungus to grow on keratin as the only source of carbon and nitrogen (Ebeling, W. et al. (1974) Eur. J. Biochem. Vol. 47(1), 91-97) Roelcke and Uhlenbruch, 1069, Z. Med. Mikrobiol. Immunol. Vol. 155(2), 156-170). Proteinase K has a specific activity of more than 30 U/mg and is thus one of the most active of the known endopeptidases (Betzel, C. et al. (1986) FEBS Lett. Vol. 197(1-2), 105-110) and unspecifically hydrolyses native and denatured proteins (Kraus, E. and Femfert, U, (1976) Hoppe Seylers Z. Physiol. Chem. Vol. 357(7):937-947).

Proteinase K from Tritirachium album Limber is translated in its natural host as a preproprotein. The sequence of the cDNA of the gene which codes for proteinase K was decoded in 1989 by Gunkel, F. A. and Gassen, H. G. (1989) Eur. J. Biochem. Vol. 179(1), 185-194. According to this the gene for prepro-proteinase K is composed of two exons and codes for a signal sequence of 15 amino acids in length, a prosequence of 90 amino acids in length and a mature proteinase K of 279 amino acids in length. A 63 bp intron is located in the region of the prosequence. The prepeptide is cleaved off during translocation into the endoplasmatic reticulum (ER). At present very little is known about the subsequent processing to form mature proteinase K with cleavage of the propeptide.

Consequently mature proteinase K consists of 279 amino acids. The compact structure is stabilized by two disulfide bridges and two bound calcium ions. This explains why proteinase K compared to other subtilisins has a considerably higher stability towards extreme pH values, high temperatures, chaotropic substances and detergents (Dolashka, P. et al. (1992) Int. J. Pept. Protein. Res. Vol. 40(5), 465-471). Proteinase K is characterized by a high thermostability (up to 65° C., Bajorath et al. (1988), Eur. J. Biochem. Vol. 176, 441-447) and a wide pH range (pH 7.5-12.0, Ebeling, W. et al. (1974) Eur. J. Biochem. Vol. 47(1), 91-97). Its activity is increased in the presence of denaturing substances such as urea or SDS (Hilz, H. et al. (1975) J. Biochem. Vol. 56(1), 103-108; Jany, K. D. and Mayer, B. (1985) Biol. Chem. Hoppe-Seyler, Vol. 366(5), 485-492).

The above-mentioned properties make proteinase K of particular interest for biotechnological applications in which an unspecific protein degradation is required. Special examples are nucleic acid isolation (DNA or RNA) from crude extracts and sample preparation in DNA analysis (Goldenberger, D. et al. (1995) PCR Methods Appl. Vol. 4(6), 368-370; U.S. Pat. No. 5,187,083; U.S. Pat. No. 5,346,999). Other applications are in the field of protein analysis such as structure elucidation.

Proteinase K is obtained commercially in large amounts by fermentation of the fungus Tritirachium album Limber (e.g. CBS 348.55, Merck strain No. 2429 or the strain ATCC 22563). The production of proteinase K is suppressed by glucose or free amino acids. Since protein-containing media also induce the expression of proteases, it is necessary to use proteins such as BSA, milk powder or soybean flour as the only nitrogen source. The secretion of the protease starts as soon as the stationary phase of growth is reached (Ebeling, W. et al. (1974) Eur. J. Biochem. Vol. 47(1), 91-97).

Since Tritirachium album Limber is consequently unsuitable for fermentation on a large scale and moreover is difficult to genetically manipulate, various attempts have been made to overexpress recombinant proteinase K in other host cells. However, no significant activity was detected in these experiments due to lack of expression, formation of inactive inclusion bodies or problems with the naturation (Gunkel, F. A. and Gassen, H. G. (1989) Eur. J. Biochem. Vol. 179(1), 185-194; Samal, B. B. et al. (1996) Adv. Exp. Med. Biol. Vol. 379, 95-104).

Moreover, Tritirachium album Limber is a slowly growing fungus which only secretes small amounts of proteases into the medium. The long fermentation period of one to two weeks is disadvantageous. In addition it is known that T. album also produces other proteases apart from proteinase K which can contaminate the preparation (Samal, B. B. et al. (1991) Enzyme Microb. Technol. Vol. 13, 66-70).

The object of the present invention is to provide a method for the economical production of recombinant proteinase K and of inactive zymogenic precursors of proteinase K that can be autocatalytically activated.

The object was achieved by providing a method for producing recombinant proteinase K in which the inactive zymogenic proform of proteinase K is produced in an insoluble form in inclusion bodies, and the zymogenic proform of proteinase K is natured and the zymogenic proform processed i.e. activated in subsequent steps. The methods for the naturation and activation of proteinase K are also a subject matter of the present invention. The present invention concerns a method for producing recombinant proteinase K characterized in that the zymogenic proform is folded by in vitro naturation and is converted by autocatalytic cleavage into the active form. The present invention concerns in particular a method for producing a recombinant proteinase K in which a zymogenic precursor of proteinase K is converted by oxidative folding from isolated and solubilized inclusion bodies into the native structure i.e. it is natured and subsequently the active proteinase K is obtained from the natively folded zymogen by autocatalytic cleavage by adding detergents.

Hence the present invention concerns a method for obtaining recombinant proteinase K by transforming a host cell with a DNA coding for the zymogenic proform of proteinase K characterized by the following process steps:
a) Culturing the said host cell under conditions which result in an expression of the DNA coding for the zymogenic proform of proteinase K such that a zymogenic precursor of proteinase K is formed in the host cell in the form of insoluble inclusion bodies.
b) Isolating the inclusion bodies, solubilizing the enzyme and naturing of the zymogenic precursor of proteinase K under conditions in which the protease part of the zymogenic precursor of proteinase K is formed.
c) Activating the proteinase K by removing the propeptide and further purification.

The DNA coding for the zymogenic proform of proteinase K corresponds to the DNA shown in SEQ ID NO: 2 or a DNA corresponding thereto within the scope of the degeneracy of the genetic code. SEQ ID NO: 2 includes the DNA sequence which codes for proteinase K and the propeptide. Furthermore the DNA can be codon-optimized for expression in a particular host. Method for codon-optimization are known to a person skilled in the art and are described in example 1. Hence the present invention concerns methods in which the host cell is transformed by a DNA which is selected from the above-mentioned group.

A proteinase K is obtained by the method according to the invention which is homogeneous and hence particularly suitable for analytical and diagnostic applications. The zymogenic proform of proteinase K according to the invention can optionally contain additional N-terminal modifications and in particular sequences which facilitate purification of the target protein (affinity tags), sequences which increase the efficiency of translation, sequences which increase the folding efficiency or sequences which result in a secretion of the target protein into the culture medium (natural presequence and other signal peptides).

Proteinase K in the sense of the invention means the sequence according to Gassen et al. (1989) shown in SEQ ID NO: 1 as well as other variants of proteinase K from Tritirachium album Limber like the amino acid sequence disclosed by Ch. Betzel et al. (Biochemistry 40 (2001), 3080-3088) and in particular proteinase T (Samal, B. B. et al. (1989) Gene Vol. 85(2), 329-333; Samal, B. B. et al. (1996) Adv. Exp. Med. Biol. Vol. 379, 95-104) and proteinase R (Samal, B. B. et al. (1990) Mol. Microbiol. Vol. 4(10), 1789-1792; U.S. Pat. No. 5,278,062) and in addition variants produced by recombinant means (as described for example in WO 96/28556). The sequence shown in SEQ ID NO: 1 comprises the signal sequence (1-15, 15 amino acids), the prosequence (16-105; 90 amino acids) and the sequence of the mature proteinase K (106-384; 279 amino acids). The amino acid sequence described by Betzel et al. (Biochemistry 40 (2001), 3080-3088) has in particular aspartate instead of a serine residue at position 207 of the active protease.

Pro-proteinase K in the sense of the invention means in particular a proteinase K whose N-terminus is linked to its prosequence. In the case of the closely related subtilisin E and other variants it is known that the prosequence has an important influence on the folding and formation of active protease (Ikemura, H. et al. (1987) Biol. Chem. Vol. 262(16), 7859-7864). In particular it is presumed that the prosequence acts as an intramolecular chaperone (Inouye, M. (1991) Enzyme Vol. 45, 314-321). After the folding it is processed to form the mature subtilisin protease by autocatalytically cleaving the propeptide (Ikemura, H. and Inouye, M. (1988) J. Biol. Chem. Vol. 263(26), 12959-12963). This process occurs in the case of subtilisin E (Samal, B. B. et al. (1989) Gene vol. 85(2), 329-333; Volkov, A. and Jordan, F. (1996) J. Mol. Biol. Vol. 262, 595-599), subtilisin BPN′ (Eder, J. et al. (1993) Biochemistry Vol. 32, 18-26), papain (Vernet, T. et al. (1991) J. Biol. Chem. Vol. 266(32), 21451-21457) and thermolysin (Marie-Claire, C. (1998) J. Biol. Chem. Vol. 273(10), 5697-5701).

If added exogenously the propeptide can also act intermolecularly in trans as a chaperone on the folding of denatured mature subtilisin protease (Ohta, Y. et al. (1991) Mol. Microbiol. Vol. 5(6), 1507-1510; Hu, Z. et al. (1996) J. Biol. Chem. Vol. 271(7), 3375-3384). The propeptide binds to the active centre of subtilisin (Jain, S. C. et al. (1998) J. Mol. Biol. Vol. 284, 137-144) and acts as a specific inhibitor (Kojima, S. et al. (1998) J. Mol. Biol. Vol. 277, 1007-1013; Li, Y. et al. (1995) J. Biol. Chem. Vol. 270, 25127-25132; Ohta, Y. (1991) Mol. Microbiol. Vol. 5(6), 1507-1510). This effect is used in the sense of the invention in order to prevent autoproteolysis of proteolysis-sensitive folding intermediates by already folded, active proteinase K during the naturation.

Only certain, usually hydrophobic core regions of the prosequence appear to be necessary for the chaperone function since mutations in wide areas have no influence on the activity (Kobayashi, T. and Inouye, M. (1992) J. Mol. Biol. Vol. 226, 931-933).

In addition it is known that propeptides can be exchanged between various subtilisin variants. Thus for example subtilisin BPN′ also recognizes the prosequence of subtilisin E (Hu, Z. et al. (1996) J. Biol. Chem. Vol. 271(7), 3375-3384).

Inclusion bodies are microscopically visible particles consisting of insoluble and inactive protein aggregates which are often formed in the cytoplasm of the host cell when heterologous proteins are overexpressed and they contain very pure target protein. Methods for producing and purifying such inclusion bodies are described for example in Creighton, T. E. (1978) Prog. Biophys. Mol. Biol. Vol. 33(3), 231-297; Marston, F. A. (1986) Biochem. J. Vol. 240(1), 1-12; Rudolph, R. (1997). Folding proteins in: Creighton, T. E. (ed.) Protein Function: A practical approach. Oxford University Press, 57-99; Fink, A. L. (1998) Fold. Des. Vol. 3(1), R9-23; and EP 0 114 506.

In order to isolate inclusion bodies the host cells are lysed after fermentation by conventional methods e.g. by ultrasound, high pressure dispersion or lysozyme. The lysis preferably takes place in an aqueous neutral to slightly acid buffer. The insoluble inclusion bodies can be separated and purified by various methods, preferably by centrifugation or filtration with several washing steps (Rudolph, R. (1997). Folding Proteins In: Creighton, T. E. (ed.) Protein Function: A practical Approach. Oxford University Press, 57-99).

The inclusion bodies obtained in this manner are then solubilized in a known manner. Denaturing agents are advantageously used for this at a concentration which is suitable for dissolving the inclusion bodies, in particular guanidinium hydro-chloride and other guanidinium salts and/or urea. In order to completely monomerize the inclusion body proteins it is also advantageous to add reducing agents such as dithiothreitol (DTT), dithioerythritol (DTE) or 2-mercaptoethanol during the solubilization in order to break possible disulfide bridges by reduction. The invention also concerns a proteinase K in which the cysteines are not reduced but are derivatized in particular with GSSG to form mixed disulfides or thiocyanates (EP 0 393 725).

Hence according to the invention the inclusion bodies are solubilized by denaturing agents and reducing agents. 6-8 M guanidinium hydrochloride or 8-10 M urea are preferred as denaturing agents and 50-200 mM DTT (dithiothreitol) or DTE (dithioerythritol) are preferred as reducing agents.

Hence the present invention concerns the prosequence according to SEQ ID NO: 1 of 90 amino acids in length (amino acids 16-105) as well as other variants which facilitate folding. It also concerns a propeptide which is added exogenously for the folding of mature proteinase K and has the functions described above.

A further subject matter of the invention is a recombinant vector which contains one or more copies of the recombinant DNA defined above. The basic vector is advantageously a plasmid preferably containing a multi-copy origin of replication, but is also possible to use viral vectors. The choice of expression vector depends on the selected host cell. Methods are used to construct the expression vector and to transform the host cell with this vector that are familiar to a person skilled in the art and are described for example in Sambrook et al. (1989), Molecular Cloning (see below). A suitable vector for expression in E. coli is for example the pKKT5 expression vector or pKK177, pKK223, pUC, pET vectors (Novagen) as well as pQE vectors (Qiagen). The expression plasmid pKKT5 is formed from pKK177-3 (Kopetzki et al., 1989, Mol. Gen. Genet. 216:149-155) by exchanging the tac promoter for the T5 promoter from pDS (Bujard et al., 1987, Methods Enzymol. 155:416-433). The EcoRI restriction endonuclease cleavage site in the sequence of the T5 promoter was removed by two point mutations.

In addition the coding DNA in the vector according to the invention is under the control of a preferably strong, regulatable promoter. A promoter that can be induced by IPTG is preferred such as the lac, lacUV5, tac or T5 promoter. The T5 promoter is especially preferred.

A host cell in the sense of the invention means any host cell in which proteins can form as inclusion bodies. It is usually a microorganism e.g. prokaryotes. Prokaryotic cells are preferred and in particular Escherichia coli. Particular preference is given to the following strains: E. coli K12 strains JM83, JM105, UT5600, RR1Δ15, DH5α, C600, TG1, NM522, M15 or the E. coli B derivatives BL21, HB101, E. coli M15 is particularly preferred.

The corresponding host cells are transformed according to the invention with a recombinant nucleic acid which encodes a recombinant zymogenic proteinase K according to SEQ ID NO:2 or with a nucleic acid which is derived from the said DNA by codon-optimization or with a DNA which is derived from the said DNA within the scope of the degeneracy of the genetic code. The E. coli host cells are preferably transformed with a codon-optimized recombinant nucleic acid coding for a recombinant zymogenic proteinase K which has been optimized for expression in Escherichia coli. Hence the present invention also concerns a suitable vector which is for example selected from the above-mentioned vectors and contains a recombinant nucleic acid that is codon-optimized for E. coli and codes for a recombinant proteinase K or a recombinant zymogenic proteinase K. Another subject matter of the invention is a host cell which is for example selected from the above-mentioned host cells which has been transformed by the above-mentioned vector.

A further subject matter of the present invention is a method for the naturation of denatured zymogenic proteinase K in which the denatured zymogenic proteinase K is transferred to a folding buffer which is characterized in that the folding buffer has the following features:
A) pH value of the buffer is in the range of 7.5 to 10.5
B) presence of low-molecular weight substances which aid folding
C) presence of a redox shuffling system
D) presence of a complexing agent at a substoichiometric concentration relative to the Ca²⁺ ions
   and wherein the method is carried out at a temperature between 0° C. and 37° C.

A low concentration of denaturing agents is preferably present during the naturation. Denaturing agents may for example be present because they are still in the reaction solution due to the prior solubilization of the inclusion bodies. The concentration of denaturing agents such as guanidine hydrochloride should be less than 50 mM.

Naturation in the sense of the invention is understood as a method in which denatured, essentially inactive protein is converted into a conformation in which the protein has the desired activity after autocatalytic cleavage and activation. This is achieved by transferring the solubilized inclusion bodies to a folding buffer while reducing the concentration of the denaturing agent. The conditions must be selected such that the protein remains in solution in this process. This can be expediently carried out by rapid dilution or dialysis against the folding buffer.

It is preferred that the folding buffer has a pH of 8 to 9. Particularly preferred buffer substances are Tris/HCl buffer and bicine buffer.

The naturation method according to the invention is preferably carried out at a temperature between 0° C. and 25° C.

The low molecular weight folding agents in the folding buffer are preferably selected from the following group of low molecular weight compounds. They can be added alone as well as in mixtures, and other substances that aid folding may be present:
L-arginine at a concentration of 0.5 to 2.0 M
Tris at a concentration of 0.5 M to 2.0 M
triethanolamine at a concentration of 0.5 M to 2.0 M
α-cyclodextrin at a concentration of 60 mM to 120 mM

Low molecular weight substances that aid folding are described for example in U.S. Pat. No. 5,593,865; Rudolph, R. (1997) Folding Proteins. In: Creighton, T. E. (ed.) Protein Function: A practical Approach. Oxford University Press, 57-99 or De Bernardez Clark, E. et al. (1999) Methods. Enzymol. Vol. 309,217-236.

The above-mentioned redox shuffling system is preferably a mixed disulfide or thiosulfonate.

Systems are for example suitable as a redox shuffling system which consist of a thiol component in an oxidized and reduced form. This allows the formation of disulfide bridges within the folding polypeptide chain during naturation by controlling the reduction potential, and on the other hand, enables the reshuffling of incorrect disulfide bridges within or between the folding polypeptide chains (Rudolph, R. (1997), see above). Preferred thiol components are for example:
glutathione in a reduced (GSH) and oxidized form (GSSH)
cysteine and cystine
cysteamine and cystamine
2-mercaptoethanol and 2-hydroxyethyldisulfide

In the naturation method according to the invention the Ca²⁺ ions are preferably present at a concentration of 1 to 20 mM. For example CaCl₂ can be added in amounts of 1 to 20 mM. The Ca²⁺ ions can bind to the calcium binding sites of the folding proteinase K.

The presence of a complexing agent preferably EDTA, in a substoichiometric concentration relative to Ca²⁺ prevents the oxidation of the reducing agent by atmospheric oxygen and protects free SH groups.

The naturation is preferably carried out at a low temperature i.e. below 20° C., preferably 10° C. to 20° C. In the method according to the invention the naturation is usually completed after a period of about 24 h to 48 h.

The present invention also concerns a folding buffer which is characterized by the following features:
A) pH value of the buffer is in the range of 7.5 to 10.5
B) presence of low-molecular weight substances which aid folding
C) presence of a redox shuffling system
D) presence of a complexing agent at a substoichiometric concentration relative to the Ca²⁺ ions.

It is especially preferred when the folding buffer has a pH of 8 to 9 and/or when the redox shuffling system is a mixed disulfide or thiosulfonate.

Another subject matter of the invention is a method for activating the natured zymogenic precursor of proteinase K. After the folding process according to the invention an inactive complex is formed from native proteinase K and the inhibitory propeptide. The active proteinase K can be released from this complex. Addition of detergents is preferred, SDS is particularly preferred at a concentration of 0.1 to 2% (w/v).

The advantages of the method disclosed here for producing recombinant proteinase K are:
1. The ability to utilize the high expression potential and the rapid and simple culture of Escherichia coli or other suitable microorganisms.
2. The possibility to genetically manipulate the recombinant DNA.
3. The uncomplicated purification after naturation.
4. The absence of eukaryotic impurities when a prokaryote is selected as a host cell.

A method would also be conceivable in which the nucleic acids which code for mature proteinase K and nucleic acids which code for the propeptide or pro-proteinase K are expressed separately in host cells and are then commonly transferred to a folding buffer for the naturation of mature proteinase K.

### DESCRIPTION OF THE FIGURES

FIG. 1:

Schematic representation of the PCR reaction to produce proteinase K fragments having an N-terminal BamHI cleavage site and an alternative enterokinase cleavage site for fusion with an N-terminal affinity tag.

FIG. 2:

Dependency of the yield of naturation on temperature.

FIG. 3:

Dependency of the yield of naturation on pH.

FIG. 4:

Dependency of the yield of naturation on redox potential.

FIG. 5:

Dependency of the yield of naturation on the arginine concentration.

FIG. 6:

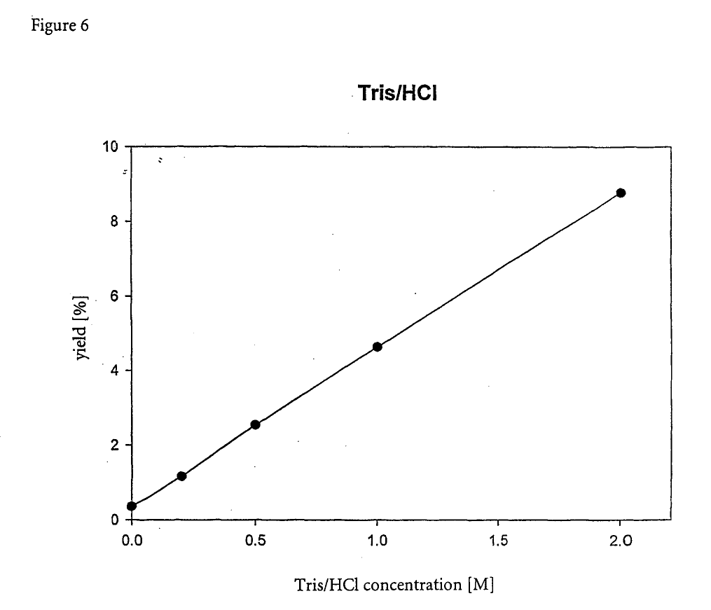

Dependency of the yield of naturation on the Tris concentration.

FIG. 7:

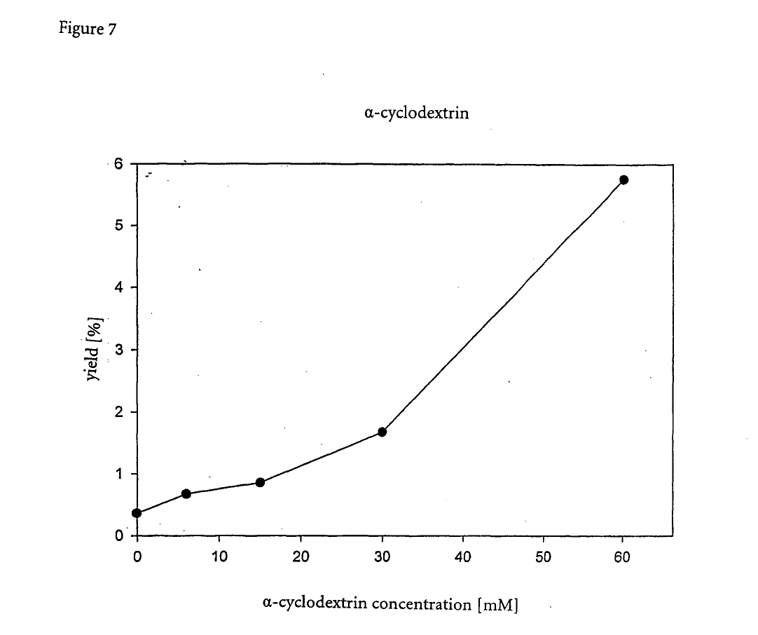

Dependency of the yield of naturation on the α-cyclodextrin concentration.

FIG. 8:

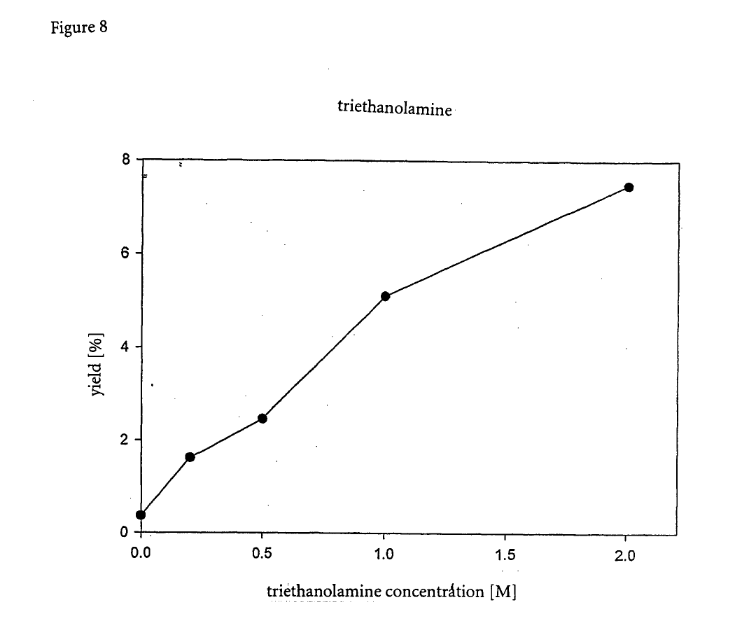

Dependency of the yield of naturation on the triethanolamine concentration.

FIG. 9:

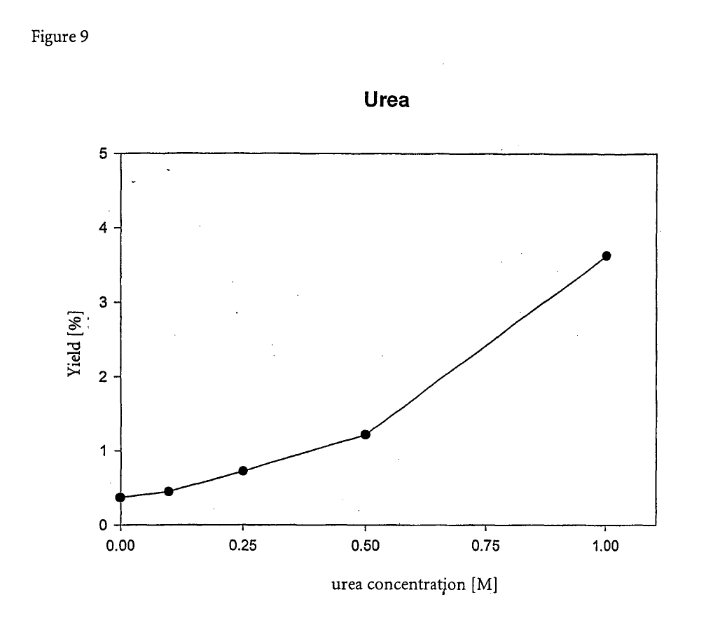

Dependency of the yield of naturation on the urea concentration.

FIG. 10:

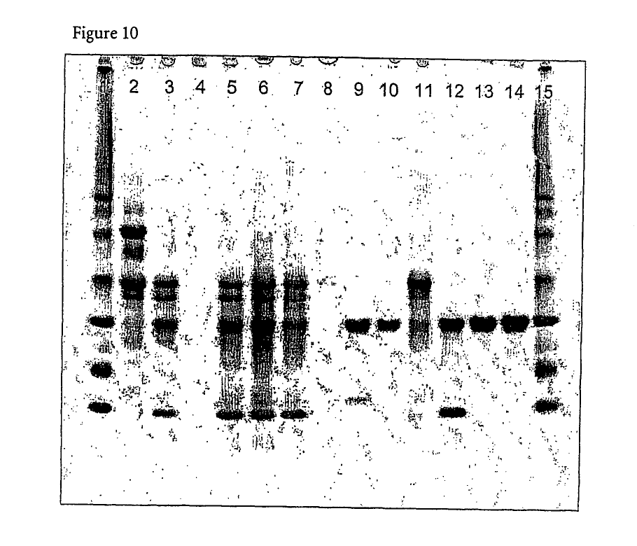

SDS polyacrylamide gel of the naturation of pro-proteinase K.

FIG. 11:

Reverse phase chromatography of natured proteinase K.

FIG. 12:

Renatured and processed proteinase K was analysed by analytical ultracentrifugation. The centrifugation was carried out at 12000 rpm, 20° C. for 63 h. The data (o) could be fitted to a homogeneous species having an apparent molecular weight of 29-490 Da. No systematic deviation was observed between the fitted and measured data (lower graph).

FIG. 13:

Determination of the Km value of natured proteinase K.

FIG. 14:

Degradation pattern of blood serum proteins by natured proteinase K.

FIG. 15:

Purification of natured proteinase K by gel filtration.

### EXAMPLE 1

Synthesis of the Gene which Codes for the Mature Form of Proteinase K.

The gene for the mature proteinase K from Tritirachium album Limber without a signal sequence and without an intron was generated by means of gene synthesis. The sequence of Gunkel, F. A. and Gassen, H. G. (1989) Eur. J. Biochem. Vol. 179(1), 185-194 of 837 bp in length (amino acids 106-384 from Swiss Prot P06873) was used as the template. A codon usage optimized for Escherichia coli was used as the basis for retranslating the amino acid sequence to optimize the expression (Andersson, S. G. E. and Kurland, C. G. (1990) Microbiol. Rev. Vol. 54(2), 198-210, Kane, J. F. Curr. Opin. Biotechnol., Vol. 6, pp. 494-500). The amino acid sequence is shown in SEQ ID NO: 1 and the nucleotide sequence is shown in SEQ ID NO: 2.

For the gene synthesis the gene was divided into 18 fragments of sense and reverse, complementary counterstrand oligonucleotides in alternating sequence (SEQ ID NO:3-20). An at least 15 bp region was attached to the 5′ end and to the 3′ end which in each case overlapped the neighbouring oligonucleotides. Recognition sites for restriction endonucleases were attached to the 5′ and 3′ ends of the synthetic gene outside the coding region for subsequent cloning into expression vectors. The oligonucleotide shown in SEQ ID NO:3 which contains an EcoRI cleavage site was used as a 5′ primer for cloning the pro-protein X gene without an N-terminal affinity tag. SEQ ID NO: 20 shows the 3′ primer containing a HindIII cleavage site. The 3′ primer contains an additional stop codon after the natural stop codon to ensure termination of the translation. The oligonucleotide with a BamHI cleavage site shown in SEQ ID NO: 23 or the oligonucleotide with a BamHI cleavage site and enterokinase cleavage site shown in SEQ ID NO: 24 was used as a 5′ primer to clone the proprotein X gene with N-terminal affinity tags and an alternative enterokinase cleavage site as described in example 3.

The oligonucleotides were linked together by means of a PCR reaction and the resulting gene was amplified. For this the gene was firstly divided into three fragments of 6 oligonucleotides each and the three fragments were linked together in a second PCR cycle. Fragment 1 is composed of the oligonucleotides shown in SEQ ID NO: 3-8, fragment 2 is composed of the oligonucleotides shown in SEQ ID NO: 9-14 and fragment 3 is composed of the oligonucleotides shown in SEQ ID NO: 15-20.

The following PCR parameters were employed

PCR reaction 1 (generation of three fragments)

| | | | |
|---|---|---|---|
| 5 min | 95° C. | | hot start |
| 2 min | 95° C. | | |
| 2 min | 42° C. | | |
| 1.5 min | 72° C. | {close oversize brace} | 30 cycles |
| 7 min | 72° C. | | final extension |

PCR reaction 2 (linkage of the fragments to form the total gene)

| | | | |
|---|---|---|---|
| 5 min | 95° C. | | hot start |
| 1.5 min | 95° C. | | |
| 2 min | 48° C. | {close oversize brace} | 6 cycles (without terminal primers) |
| 2 min | 72° C. | | |

addition of terminal primers

| | | | |
|---|---|---|---|
| 1.5 min | 95° C. | | |
| 1.5 min | 60° C. | {close oversize brace} | 25 cycles (with terminal primers) |
| 2 min | 72° C. | | |
| 7 min | 72° C. | | final extension |

### EXAMPLE 2

Cloning of the Synthetic Proteinase K Fragment from the Gene Synthesis

The PCR mixture was applied to an agarose gel and the ca. 1130 bp PCR fragment was isolated from the agarose gel (Geneclean II Kit from Bio 101, Inc. CA USA). The fragment was cleaved for 1 hour at 37° C. with EcoRI and HindIII restriction endonucleases (Roche Diagnostics GmbH, Germany). At the same time the pUC18 plasmid (Roche Diagnostics GmbH, Germany) was cleaved for 1 hour at 37° C. with EcoRI and HindIII restriction endonucleases, the mixture was separated by agarose gel electrophoresis and the 2635 bp vector fragment was isolated. Subsequently the PCR fragment and the vector fragment were ligated together using T4 DNA ligase. For this 1 μl (20 ng) vector fragment and 3 μl (100 ng) PCR fragment, 1 μl 10× ligase buffer (Maniatis, T., Fritsch, E. F. and Sambrook, T. (1989). Molecular Cloning: A laboratory manual. 2^{nd} ed., Cold Spring Harbor Press, Cold Spring Harbor, N.Y.), 1 μl T4 DNA ligase, 4 μl sterile redistilled H₂O were pipetted, carefully mixed and incubated overnight at 16° C.

The cloned gene was examined by restriction analysis and by multiple sequencing of both strands. The sequence is shown in SEQ ID NO: 2.

a) Construction of the pPK-1 Expression Plasmid

In order to express proteinase K, the structural gene was cloned into the pKKT5 expression vector in such a manner that the structural gene is inserted in the correct orientation under the control of a suitable promoter, preferably a promoter that can be induced by IPTG such as the lac, lacUV5, tac or T5 promoter, particularly preferably the T5 promoter. For this purpose the structural gene for proteinase K was cleaved from the plasmid pUC18 by EcoRI and HindIII, the restriction mixture was separated by agarose gel electrophoresis and the ca. 1130 bp fragment was isolated from the agarose gel. At the same time the expression plasmid pKKT5 was cleaved with EcoRI and HindIII, the restriction mixture was separated by agarose gel electrophoresis and the ca. 2.5 kbp vector fragment was isolated from the agarose gel. The fragments obtained in this manner were ligated together as described above. The correct insertion of the gene was checked by sequencing.

b) Transformation of the Expression Plasmid pPK-1 in Various E. coli Expression Strains

The expression vector was transformed in various expression strains that had been previously transformed with the plasmid pREP4 and/or pUBS520. The plasmid pREP4 contains a gene for the lacI repressor that should ensure a complete suppression of the expression before induction. The plasmid pUBS520 (Brinkmann, U. et al. (1989) Gene Vol. 85(1), 109-114) also contains the lacI repressor and additionally the dnaY gene which codes for the tRNA which is necessary to translate the rare arginine codons AGA and AGG in E. coli. Competent cells of various E. coli strains were prepared according to the method of Hanahan, D. (1983) J. Mol. Biol. Vol. 166, 557-580. 100 μl cells prepared in this manner was admixed with 20 ng isolated pPK-1 plasmid DNA. After 30 min incubation on ice, they were heat-shocked (90 sec at 42° C.) and then incubated for 2 min on ice. Subsequently the cells were transferred to 1 ml SOC medium and incubated for 1 hour at 37° C. while shaking for the phenotypic expression. Aliquots of this transformation mixture were plated out on LB plates containing ampicillin as a selection marker and incubated for 15 hours at 37° C. Preferred strains are E. coli K12-strains JM83, JM105, UT5600, RR1Δ15, DH5α, C600, TG1, NM522, M15 or the E. coli B derivatives BL21, HB101; E. coli M15 is particularly preferred.

### EXAMPLE 3

Cloning of an N-Terminal Affinity Tag

In order to insert an N-terminal affinity tag, a BamHI cleavage site was inserted before the 5′ end of the gene for pro-proteinase K. This was achieved by PCR using the product obtained in example 1 as a template and the oligonucleotides described in SEQ ID NO:20, 23 and 24 as primers. The primer described in SEQ ID NO:23 contains a BamHI cleavage site upstream of the 5′ region of pro-proteinase K, the primer described in SEQ ID NO:24 additionally contains an enterokinase cleavage site directly in front of the first codon of the prosequence. SEQ ID NO:20 shows the 3′ primer that was also used in example 1 with a HindIII cleavage site. The resulting PCR products were isolated as described above, digested with BamHI and HindIII and purified by agarose electrophoresis.

The affinity tag was inserted by means of a synthetic linker composed of two complementary oligonucleotides in such a manner that an EcoRI cleavage site was formed at the 5′ end and a BamHI cleavage site was formed at the 3′ end without further restriction digestion. For a His tag the sense strand had the sequence shown in SEQ ID NO:21 and the antisense strand had the sequence shown in SEQ ID NO.22. The linker coded for a hexa-His tag with an N-terminal RGS motif. The BamHI cleavage site between the linker and pro-proteinase K is translated into a Gly-Ser linker. In order to anneal the linker, the two oligonucleotides (SEQ ID NO:21 and 22) were heated for 5 min to 95° C. in equimolar amounts (50 pmol/μl each) and subsequently cooled at 2° C. per min to room temperature. As a result the annealing of the complementary oligonucleotides should be as complete as possible.

The linker was ligated with the BamHI/HindIII-digested PCR product. (Rapid Ligation Kit from Roche Diagnostics GmbH, Germany) and purified by agarose gel electrophoresis (QIAquick gel extraction Kit from Qiagen, Germany). The resulting ligation product was ligated into an expression vector analogously to example 2b via the EcoRI and HindIII overhangs and transformed correspondingly in expression strains.

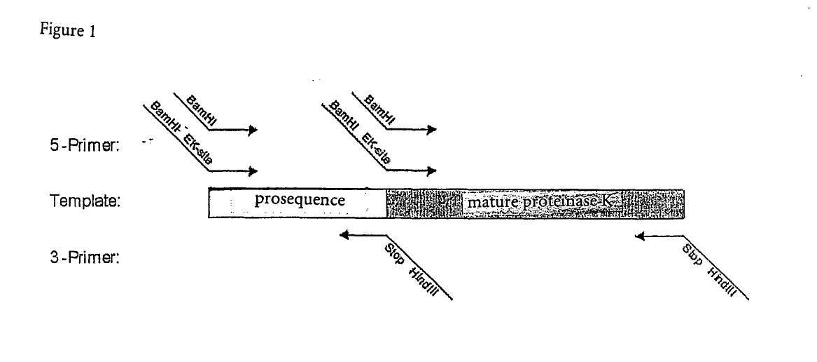

This module system enables various affinity tags that are coded by the synthetic linker to be fused to the structural gene for pro-proteinase K. An enterokinase cleavage site can be alternatively inserted between the tag and propeptide by suitable selection of the corresponding 5′ primer if a subsequent removal of the tag is desired. Furthermore a certain region of the proteinase K gene such as the mature proteinase K or the propeptide can be amplified by suitable selection of the overlapping regions of the PCR primers (FIG. 1).

### EXAMPLE 4

Expression of Proteinase K in Escherichia coli

Since proteinase K is a very active unspecific protease, it is preferable to express it in an inactive form preferably as inclusion bodies.

In order to express the gene which codes for proteinase K, 3 ml Lbₐₘₚ medium was inoculated with plasmid-containing clones and incubated at 37° C. in a shaker.
LB medium: 10 g tryptone
   10 yeast extract
   5 g NaCl
   make up to a final volume of 1 l with distilled H₂O, adjust to pH 7.0 with NaOH
   addition of antibiotics (100 μg/ml ampicillin) directly before inoculation

The cells were induced with 1 mM IPTG at an optical density of 0.5 at 550 nm and incubated for 4 h at 37° C. in a shaker. Subsequently the optical density of the individual expression clones was determined, an aliquot corresponding to an OD₅₅₀ of 3/ml was removed and the cells were centrifuged (10 min 6000 rpm, 4° C.). The cells were resuspended in 400 μl TE buffer, lysed by ultrasound and the soluble protein fraction was separated from the insoluble protein fraction by centrifugation (10 min, 14,000 rpm, 4° C.).

| | |
|---|---|
| TE buffer: | 50 mM Tris/HCl |
| | 50 mM EDTA |
| | pH 8.0 (at RT) |

Application buffer containing SDS and β-mercaptoethanol was added to all fractions and the proteins were denatured by heating (5 min 95° C.). Subsequently 10 μl aliquots were analysed by means of a 12.5% analytical SDS gel (Laemmli, U.K. (1970) Nature Vol. 227(259), 680-685). A very strong expression in the form of insoluble protein aggregates (inclusion bodies) was observed for the clones of mature proteinase K as well as for the clones of pro-proteinase K. Accordingly no proteinase K activity was measured.

### EXAMPLE 5

Isolation of the Inclusion Bodies

The inclusion bodies were prepared by known methods (Rudolph, R. (1997) see above).

For the cell lysis, 10 g wet biomass was in each case resuspended in 50 ml 100 mM Tris/HCl pH 7.0, 1 mM EDTA. Afterwards 15 mg lysozyme was added, incubated for 60 min at 4° C. and the cells were subsequently lysed by high pressure (Gaulin cell lysis apparatus). The DNA was digested for 30 min at RT by adding 3 mM MgCl₂ and 10 μg/ml DNase to the crude extract. The insoluble cell components which contain the inclusion bodies were separated by centrifugation (30 min 20,000 g) and washed once with washing buffer 1 and three times with washing buffer 2.

| | |
|---|---|
| washing buffer 1: | 100 mM Tris/HCl |
| | 20 mM EDTA |
| | 2% (v/v) Triton X-100 |
| | 0.5 M NaCl |
| | pH 7.0 (RT) |
| washing buffer 2: | 100 mM Tris/HCl |
| | 1 mM EDTA |
| | pH 7.0 (RT) |

The pellet of the last washing step constitutes the crude inclusion bodies which already contain highly pure target protein.

### EXAMPLE 6

Solubilization of Inclusion Bodies

a) Solubilization While Reducing with Cysteines

1 g crude inclusion bodies was suspended in 10 ml solubilization buffer and incubated for 2 h at RT while stirring gently.

| | |
|---|---|
| Solubilization buffer: | 100 mM Tris/HCl |
| | 6.0 M guanidinium hydrochloride |
| | 100 mM DTT |
| | pH 8.0 (RT) |

The solubilisate was titrated to pH 3 with 25% HCl and dialysed twice for 4 h at RT against 500 ml 6 M guanidine hydrochloride pH 3 and then overnight at 4° C. against 1000 ml guanidine hydrochloride pH 7. The protein concentration was determined by the Bradford method (Bradford, 1976) using a calculated extinction coefficient at 280 nm and was between 10 and 20 mg/ml. The number of free cysteines was determined according to the Ellman method. In accordance with the sequence 5 mol free cysteines per mol proteinase K were found. The purity of the solubilized inclusion bodies was determined by 12.5% SDS PAGE and quantification of the bands after Coomassie staining.

b) Solubilization with Derivatization of the Cysteines to Form Mixed Disulfides Using Glutathione. 1 g Crude Inclusion Bodies Were Suspended in 10 ml Solubilization Buffer.

| | |
|---|---|
| Solubilization buffer: | 100 mM Tris/HCl |
| | 6.0 M guanidine hydrochloride |
| | 1 mM DTT |
| | pH 8.0 (RT) |

After 15 min incubation at RT while stirring gently, during which a catalytic amount of reduced cysteines was formed due to the small amounts of DTT, 100 mM GSSG was added, the pH was adjusted to 8.0 and it was incubated for a further 2 h at RT while stirring gently.

Further treatment as described under a).

### EXAMPLE 7

Optimization of the Naturation of Pro-Proteinase K

Various parameters were varied in order to optimize the yield in the folding and processing of pro-proteinase K from the solubilisates prepared in example 6a). For all preparations the stated folding buffer was filtered, degassed, gassed with N₂ and incubated at the desired temperature. The redox shuffling system was not added until shortly before the start of the folding reaction and the pH was readjusted. The folding was initiated by adding the solubilized inclusion bodies while rapidly mixing. The volume of the folding mixtures was 1.8 ml in 2 ml glass tubes with a screw cap. The yield was analysed by an activity test using the chromogenic substrate Suc-Ala-Ala-Pro-Phe-pNA from the Bachem Company (Heidelberg). 100 mM Tris/HCl, 5 mM CaCl₂, pH 8.5 at 25° C. was used as the test buffer. The concentration of the peptide in the test was 2 mM from a 200 mM stock solution in DMSO. In order to activate the renaturate, 0.1% SDS was added to the sample (see example 8). The absorbance at 410 nm was measured over a period of 20 min and the activity was calculated from the slope.

The following parameters were varied:

a) Temperature and Time

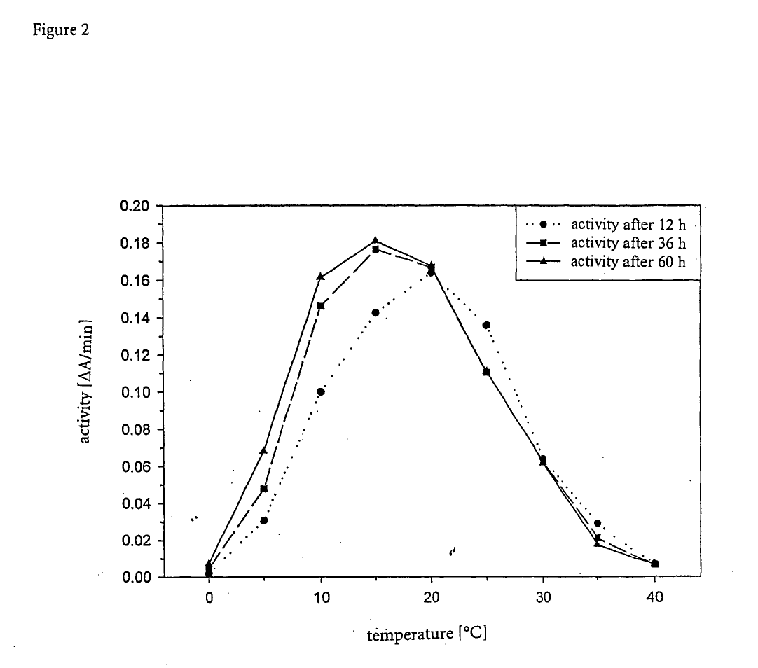

The folding buffer containing 100 mM Tris, 1.0 mM L-arginine, 10 mM CaCl₂ was equilibrated at various temperatures. After adding 3 mM GSH and 1 mM GSSG the pH was readjusted at the corresponding temperature. The reaction was started by adding 50 μg/ml pro-proteinase K. After 12 h, 36 h and 60 h, aliquots were removed and tested for activity. The results are shown in FIG. 2.

b) pH Value

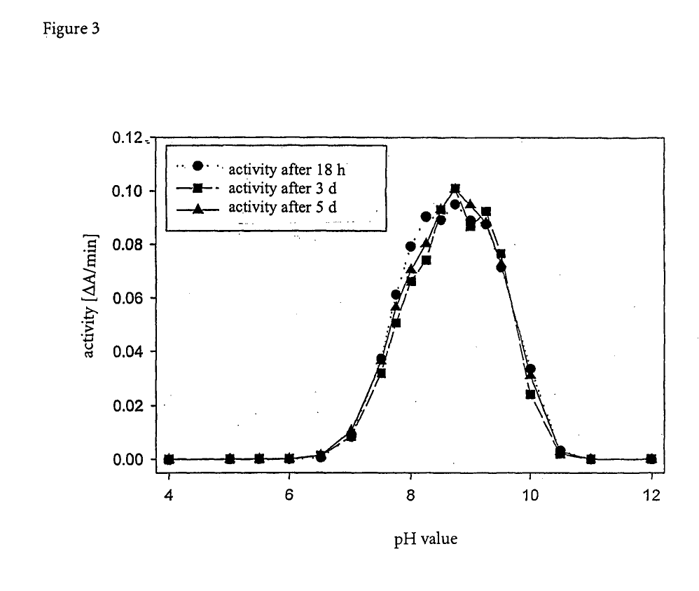

A universal buffer containing 50 mM citrate, 50 mM MES, 50 mM bicine, 500 mM arginine, 2 mM CaCl₂ and 1 mM EDTA was incubated at 15° C. and 3 mM GSH and 1 mM GSSG were added. The pH was readjusted in a range between pH 4.0 and pH 12.0 and the folding reaction was started by adding 50 μg/ml pro-proteinase K inclusion bodies. The activity measured after 18 h, 3 d and 5 d is shown in FIG. 3.

c) Redox Potential

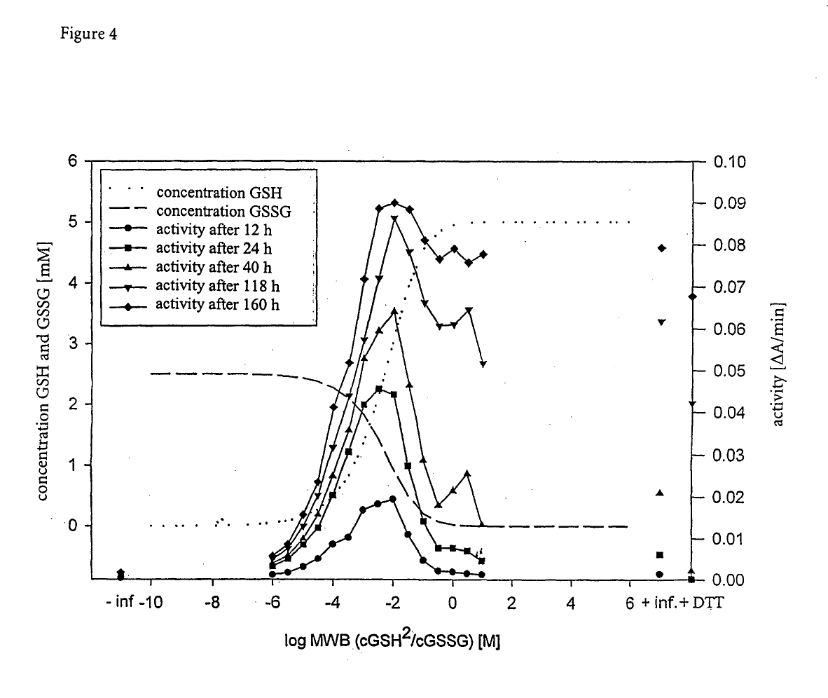

Various redox potentials were set in a renaturation buffer containing 1.0 M L-arginine, 100 mM bicine, 2 mM CaCl₂ and 10 mM CaCl₂ by mixing various ratios of oxidized and reducing glutathione. The protein concentration in the folding mixture was 50 μg/ml. The folding was carried out at 15° C. The concentrations of GSH and GSSG are shown in table 1, the measurements are shown in FIG. 4.

TABLE 1
concentrations of GSH and GSSG at the various redox potentials.

| Redox potential | | |
|---|---|---|
| (log(cGSH2/cGSSG) [M] | c(GSH) [mM] | c(GSSG) [mM] |
| −00 | 0 | 2.500 |
| −6.000 | 0.050 | 2.475 |
| −5.500 | 0.088 | 2.456 |
| −5.000 | 0.156 | 2.422 |
| −4.500 | 0.273 | 2.363 |
| −4.000 | 0.476 | 2.262 |
| −3.500 | 0.814 | 2.093 |
| −3.000 | 1.351 | 1.825 |
| −2.500 | 2.130 | 1.435 |
| −2.000 | 3.090 | 0.955 |
| −1.500 | 3.992 | 0.504 |
| −1.000 | 4.580 | 0.210 |
| −0.500 | 4.851 | 0.074 |
| 0.000 | 4.951 | 0.025 |
| +0.500 | 4.984 | 7.856e−3 |
| +1.000 | 4.995 | 2.495e−3 |
| +00 | 5.000 | 0 |

d) Solvent Additives that Promote Folding

Various substances were examined for their ability to increase the folding yield of proteinase K. For this purpose solutions containing the substances at various concentrations were prepared and admixed with 2 mM CaCl₂, 1 mM EDTA and 100 mM bicine. The pH was adjusted to pH 8.75 at the folding temperature of 15° C.

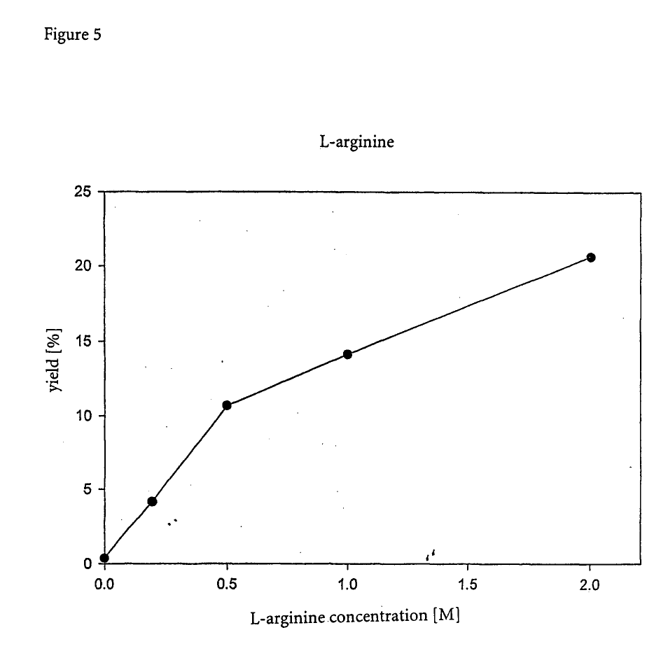

The protein concentration was 50 μg/ml. FIG. 5 shows the relative yields of active proteinase K in relation to the concentration of the selected buffer additive.

### EXAMPLE 8

Activation of the Natured Pro-Proteinase K

After naturation of pro-proteinase K by the method according to the invention it was found to have no activity or only a very slight activity. Chromatographic methods and SDS-PAGE showed that mature proteinase K is already present but is still associated in a complex with the propeptide. This can be separated in a method which is referred to here as activation and is also a subject matter of the invention.

In this example SDS is added at a concentration of 2% (v/v) to the folding mixture and subsequently the folding additive and the SDS are removed by dialysis. Alternatively SDS could also be added after removing the additives by dialysis. In all cases full activity of proteinase K was detected.

### EXAMPLE 9

Characterization of the Folding Product

The proteinase K natured and activated by the method according to the invention was further characterized by various methods.

a) Analysis of Purity and Molecular Weight Determination by SDS Polyacrylamide Gel Electrophoresis

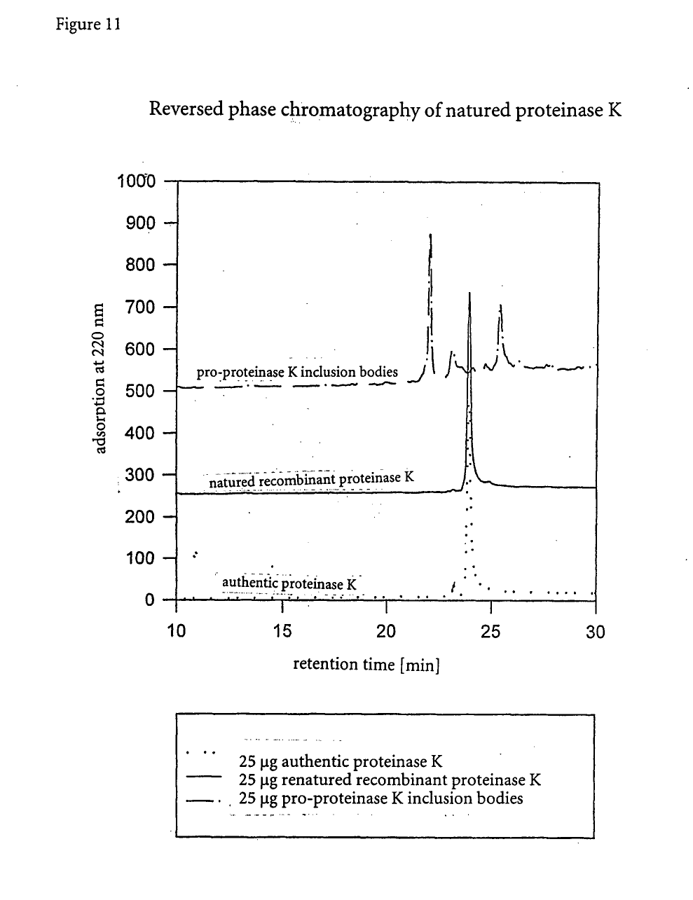

Aliquots from various steps in the maturation process and the final product, the folded and activated recombinant proteinase K were applied to a 12.5% SDS polyacrylamide gel. The samples each contained 10 mM DTT or 1% (v/v) 2-mercaptoethanol. The recombinant proteinase K prepared by the method according to the invention had no significant contamination and runs identically with the authentic proteinase K at an apparent molecular weight of ca. 30 kDa (see FIG. 11).

b) Analysis of Purity Using RP-HPLC

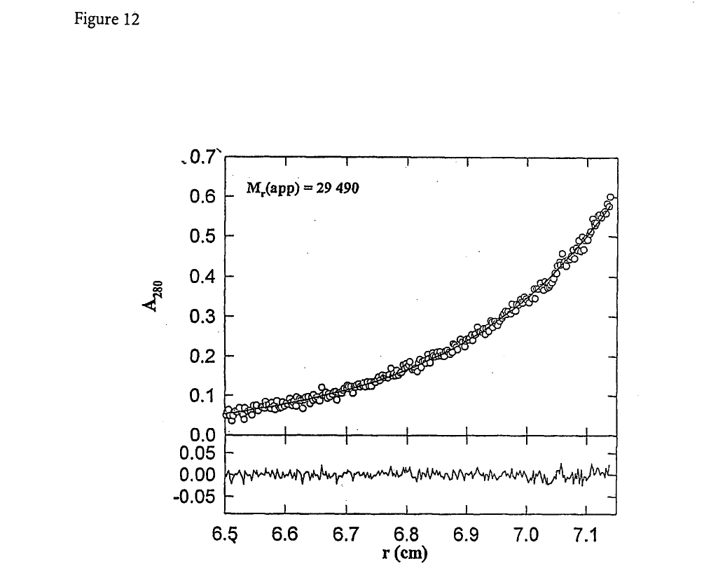

The folded and activated proteinase K and the authentic proteinase K from T. album and the pro-proteinase K inclusion bodies were analysed by means of reversed phase HPLC. A Vydac C4 column having the dimensions 15 cm×4.6 cm diameter was used. The samples were eluted with an acetonitrile gradient of 0% to 80% in 0.1% TFA. The folding product exhibits mobility properties that are identical to the authentic proteinase K used as a standard (see FIG. 12).

c) Analytical Ultracentrifugation

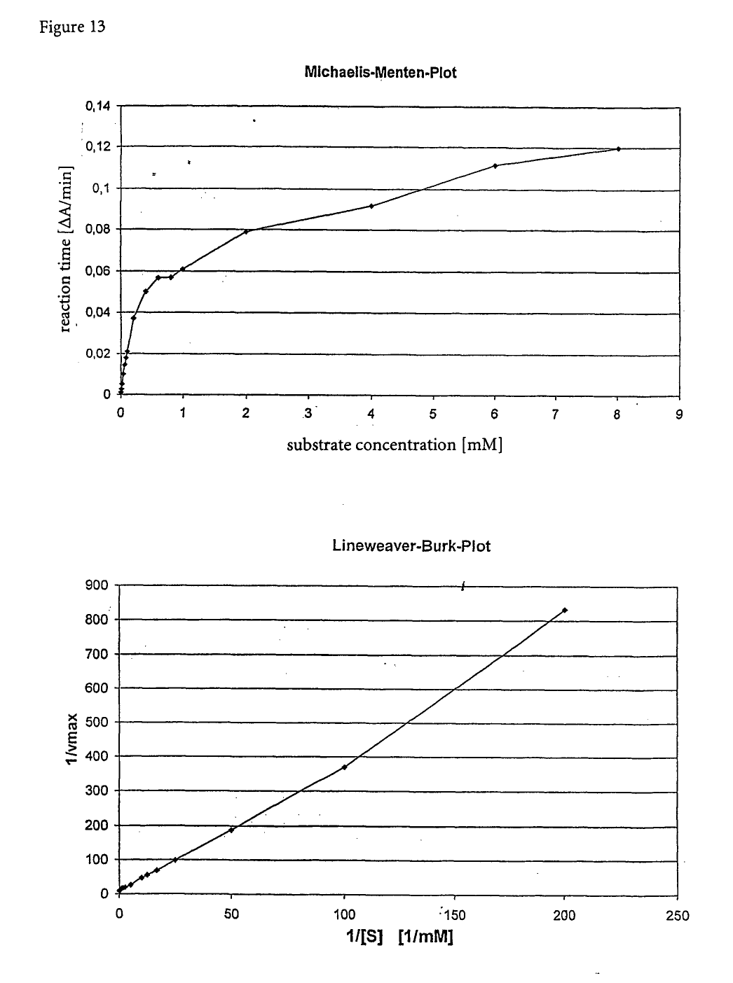

In order to analyse whether the renatured and processed proteinase K is present in a monomeric form without propeptide, the protein was examined by means of analytical ultracentrifugation. The molecular weight was determined to be 29490 Da and corresponds to the mass of the monomeric mature proteinase K within the limits of error of this method (see FIG. 13). Hence this showed that the propeptide was quantitatively cleaved by activation of the proteinase K.

d) N-Terminal Sequence Analysis

In order to examine whether the propeptide was cleaved at the correct cleavage site the natured and activated recombinant proteinase K was subjected to a sequence analysis. For this the folding product was desalted by RP-HPLC as described in example 9b) and the first 6 residues were examined by N-terminal sequencing. The result (AAQTNA) agrees with the authentic N-terminus of mature proteinase K.

e) Activity and Kₘ Value

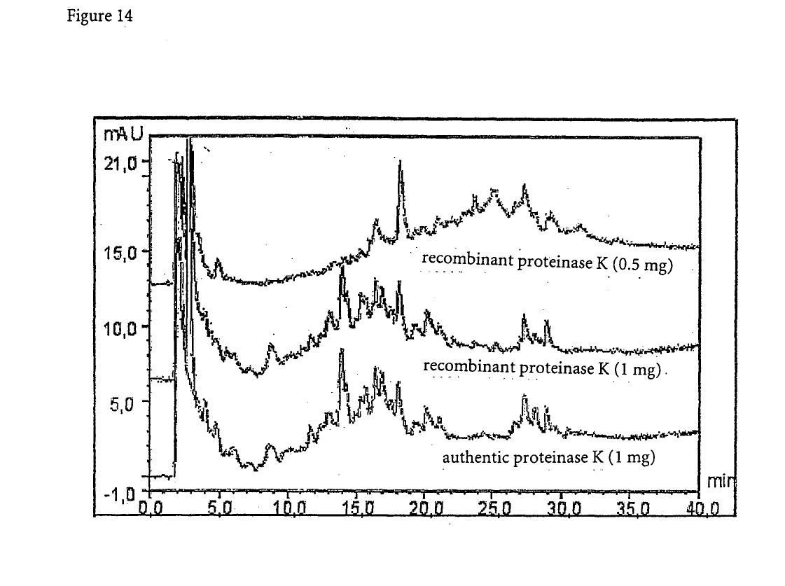

the Kₘ value of the folded and activated proteinase K was compared with that of the authentic proteinase K. The tetrapeptide Suc-Ala-Ala-Pro-Phe-pNA was used as a substrate. The test was carried out in 2.0 ml 50 mM Tris, pH 8.5 containing 1 mM CaCl₂ at 25° C. The hydrolysis of the peptide was monitored spectroscopically at 410 nm. A Kₘ value of 0.16 mM was found for the recombinant proteinase K which corresponded very well with the Kₘ value of authentic proteinase K (see FIG. 14).

f) Degradation Pattern of Blood Serum Proteins

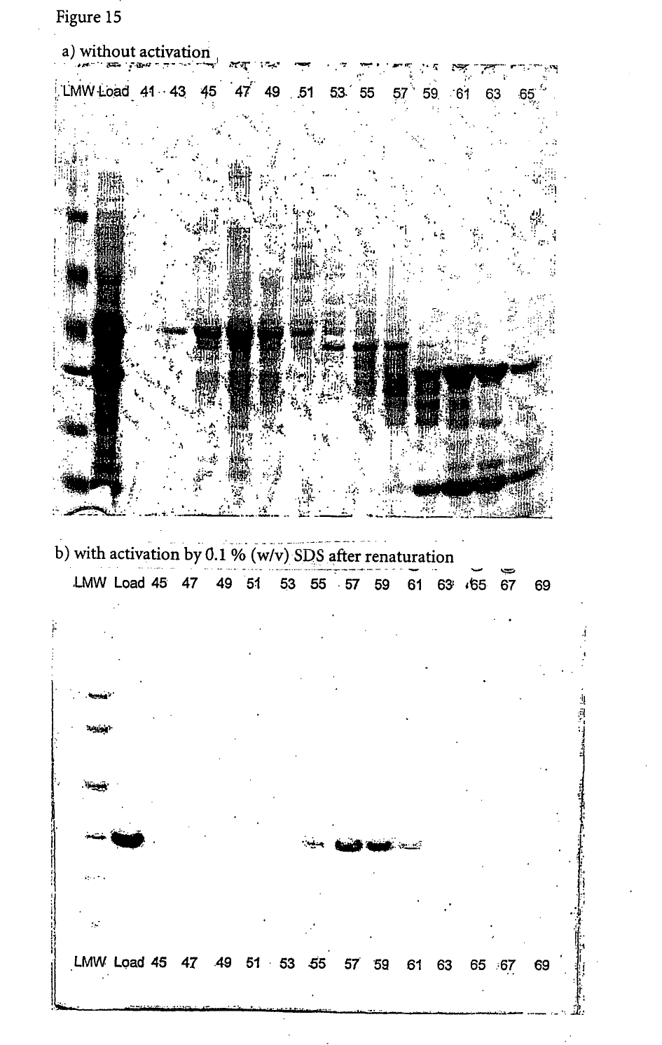

In an additional test to characterize the activity, the cleavage pattern of blood serum proteins was examined. For this a defined amount of blood serum proteins was digested with 1 μg recombinant proteinase K or the same amount of authentic proteinase K. The cleavage pattern was analysed by means of RP-HPLC under identical conditions as described in example 9b). FIG. 15 shows that the recombinant and the authentic proteinase K result in an identical degradation pattern.

### EXAMPLE 10

Purification of the Folding Product

The recombinant pro-proteinase K natured by the method according to the invention was purified by gel filtration. As described in FIG. 11 the concentrated naturation solution was separated on a Superdex 75 pg after naturation in a first run without prior activation and in a second run with prior activation using 0.15% (w/v) SDS (30 min, 4° C.). 100 mM Tris/HCl, 150 mM NaCl pH 8.75 (4° C.) was used as the mobile buffer. The application volume was 10 ml at a column volume of 1200 ml and a flow rate of 5 ml/min. After completion of the application, 14 ml fractions were collected. Aliquots of the fractions were precipitated with trichloroacetic acid, washed and taken up in Laemmli sample buffer containing 10 mM DTT. The samples were applied to a 12.5% SDS polyacrylamide gel which was stained after the run with Coomassie blue R250.

In the first run without activation a non-processed recombinant pro-proteinase K is seen in a first peak which probably runs in the form of microaggregates in the exclusion volume. In a second peak one observes processed recombinant proteinase K which co-elutes with the propeptide which is non-covalently bound and acts as an inhibitor. As a result no activity is found without prior activation. Only after adding SDS to the fractions did the second peak exhibit significant proteinase K activity (not shown).

The second run in which the folded recombinant proteinase K was previously activated with SDS only shows one peak which elutes after an identical volume like proteinase K under the same conditions (not shown). On the SDS gel one sees clean mature recombinant proteinase K without propeptide in this peak. All impurities and the propeptide appear to have already been digested in the applied mixture by the activated recombinant proteinase K. As expected the fractions of the proteinase K peak exhibited activity without further activation with SDS. The recombinant proteinase K purified in this manner appears to be almost 100% pure on the SDS gel and shows an identical migration behaviour to the authentic proteinase K (FIG. 16).

What is claimed is:

1-22. (canceled)

23. A method for the naturation of denatured zymogenic proteinase K comprising transferring the denatured zymogenic proteinase K to a folding buffer, the buffer comprising low molecular weight substances which aid folding, a redox shuffling system, and a complexing agent at a substoichiometric concentration relative to any Ca²⁺ ions that are present, the buffer having a pH of 7.5 to 10.5 and the method being carried out at a temperature between 0° C. and 37° C.

24. The method of claim 23 wherein the redox shuffling system comprises mixed disulfides or thiosulfonates.

25. The method of claim 23 wherein the pH range is 8 to 9.

26. The method of claim 23 wherein the temperature is between 0° C. and 25° C.

27. The method of claim 23 wherein the buffer further comprises denaturing agents at a concentration of less than 50 mM.

28. The method of claim 23 wherein the low molecular weight substances are selected from the group consisting of L-arginine at a concentration of 0.5 to 2.0 M, Tris at a concentration of 0.5 M to 2.0 M, triethanolamine at a concentration of 0.5 M to 2.0 M, and α-cyclodextrin at a concentration of 60 mM to 120 mM.

29. The method of claim 23 wherein the Ca²⁺ ion concentration is 1 to 20 mM.

30. The method of claim 23 wherein the denatured zymogenic proteinase K is transferred to the folding buffer while reducing the concentration of denaturing agents that may be present.

31. A folding buffer comprising low molecular weight substances which aid folding, a redox shuffling system, and a complexing agent at a substoichiometric concentration relative to any Ca²⁺ ions that are present, the buffer having a pH value in the range of 7.5 to 10.5.

32. The buffer of claim 31 wherein the pH value is 8 to 9 and the redox shuffling system comprises mixed disulfides or thiosulfonates.

33. A method for activating a natured zymogenic precursor of active proteinase K comprising adding a detergent to an inactive complex comprising a native proteinase K and an inhibitory propeptide of the active proteinase K, thereby releasing the active proteinase K from the inactive complex.

34. The method of claim 33 wherein the detergent is SDS at a concentration of 0.1 to 2% (w/v).

35. A method for producing active recombinant proteinase K comprising
(a) producing an inactive zymogenic proform of proteinase K in an inclusion body,
(b) naturing in vitro the zymogenic proform of proteinase K, and
(c) activating the zymogenic proform by autocatalytic cleavage, thereby converting it to the active proteinase K.

36. The method of claim 35 wherein the naturing step comprises transferring the denatured zymogenic proteinase K to a folding buffer, the buffer comprising low molecular weight substances which aid folding, a redox shuffling system, and a complexing agent at a substoichiometric concentration relative to any Ca²⁺ ions that are present, the buffer having a pH of 7.5 to 10.5 and the naturing step being carried out at a temperature between 0° C. and 37° C.

37. The method of claim 35 wherein the inclusion body is solubilized by a denaturing agent and a reducing agent.

38. The method of claim 37 wherein the denaturing agent is guanidinium hydrochloride at a concentration of 6-8 M or urea at a concentration of 8-10 M and the reducing agent is DTT or DTE at a concentration of 50-200 mM.

39. A method for producing active recombinant proteinase K comprising
(a) transforming a host cell with a vector containing a DNA sequence coding for a zymogenic precursor of proteinase K
(b) expressing the zymogenic precursor in inclusion bodies,
(c) isolating the inclusion bodies and solubilizing the zymogenic precursor,
(d) naturing the zymogenic precursor with a folding buffer comprising low molecular weight substances which aid folding, a redox shuffling system, and a complexing agent at a substoichiometric concentration relative to any Ca²⁺ ions that are present, the buffer having a pH of 7.5 to 10.5 and the naturing step being carried out at a temperature between 0° C. and 37° C., and
(e) activating the zymogenic precursor by autocatalytic cleavage, thereby converting it to the active proteinase K

40. The method of claim 39 wherein the host cell is a prokaryotic cell.

41. The method of claim 39 wherein the host cell is Escherichia coli.

42. A codon-optimized recombinant nucleic acid comprising DNA coding for a recombinant zymogenic proteinase K which has been optimized for expression in Escherichia coli.

43. A vector containing the recombinant nucleic acid of claim 42.

44. A host cell transformed with the vector of claim 43.


SEQUENCE LISTING (24 sequences)

SEQ ID NO: 1 - protein, 384 aa, Tritirachium album limber
MRLSVLLSLLPLALGAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 2 - DNA, 1116 bp, Tritirachium album limber
atggctcctgccgttgagcagcgctccgaggctgctcctctgatcgaggcccgcggcgagatggttgccaacaagtacatcgtcaagttcaaggagggtagcgctctttccgctctggatgctgccatggagaagatctctggcaagcccgaccacgtctacaagaacgtcttcagcggtttcgctgcgaccctggacgagaacatggttcgggttctccgcgcccaccccgatgttgagtacatcgagcaggatgctgttgtcaccatcaacgctgcgcagaccaacgctccctggggcctggctcgcatctccagcaccagccccggtacctctacctactactatgacgaatctgccggccaaggctcctgcgtctacgtgatcgacaccggtatcgaggcatcgcaccccgagttcgagggtcgtgcccagatggtcaagacctactactactccagtcgcgacggtaacggtcacggcacccactgcgctggtaccgttggctcccgtacctacggtgtcgccaagaagacccagctgttcggtgtcaaggtcctggatgacaacggcagtggccagtactccaccatcatcgccggtatggacttcgttgccagcgacaagaacaaccgcaactgccccaaaggtgtcgttgcctccttatccctgggcggtggttactcctcctccgtgaacagcgccgctgcccgcctccagagctctggtgtcatggtcgccgtcgctgccggtaacaacaacgctgacgcccgcaactactcccctgcttctgagccctcggtctgcaccgtcggtgcttctgaccgctacgaccgccgctccagcttctccaactacggcagcgttttggacatcttcggccctggtaccagcatcctctccacctggatcggcggcagcacccgctccatctctggtacctccatggctactccccacgttgccggtctcgctgcctacctcatgactcttggaaagactaccgccgccagcgcttgccgatacattgccgacaccgccaacaagggcgacttaagcaacattcccttcggcactgtcaacttgcttgcctacaacaactaccaggcttaatga

SEQ ID NO: 3 - DNA, 83 bp, Artificial Sequence
  Description of Artificial Sequence Primer
atatgaattcatggctcctgccgttgagcagcgctccgaggctgctcctctgatcgaggcccgcggcgagatggttgccaaca

SEQ ID NO: 4 - DNA, 80 bp, Artificial Sequence
  Description of Artificial Sequence Primer
atcttctccatggcagcatccagagcggaaagagcgctaccctccttgaacttgacgatgtacttgttggcaaccatctc

SEQ ID NO: 5 - DNA, 80 bp, Artificial Sequence
  Description of Artificial Sequence Primer
tgccatggagaagatctctggcaagcccgaccacgtctacaagaacgtcttcagcggtttcgctgcgaccctggacgaga

SEQ ID NO: 6 - DNA, 64 bp, Artificial Sequence
  Description of Artificial Sequence Primer
tgctcgatgtactcaacatcggggtgggcgcggagaacccgaaccatgttctcgtccagggtcg

SEQ ID NO: 7 - DNA, 65 bp, Artificial Sequence
  Description of Artificial Sequence Primer
tgagtacatcgagcaggatgctgttgtcaccatcaacgctgcgcagaccgctgcgcagaccaacg

SEQ ID NO: 8 - DNA, 70 bp, Artificial Sequence
  Description of Artificial Sequence Primer
agtaggtagaggtaccggggctggtgctggagatgcgagccaggccccagggagcgttggtctgcgcagc

SEQ ID NO: 9 - DNA, 80 bp, Artificial Sequence
  Description of Artificial Sequence Primer
gtacctctacctactactatgacgaatctgccggccaaggctcctgcgtctacgtgatcgacaccggtatcgaggcatcg

SEQ ID NO: 10 - DNA, 81 bp, Artificial Sequence
  Description of Artificial Sequence Primer
ttaccgtcgcgactggagtagtagtaggtcttgaccatctgggcacgaccctcgaactcggggtgcgatgcctcgataccg

SEQ ID NO: 11 - DNA, 78 bp, Artificial Sequence
  Description of Artificial Sequence Primer
ccagtcgcgacggtaacggtcacggcacccactgcgctggtaccgttggctcccgtacctacggtgtcgccaagaaga

SEQ ID NO: 12 - DNA, 73 bp, Artificial Sequence
  Description of Artificial Sequence Primer
atggtggagtactggccactgccgttgtcatccaggaccttgacaccgaacagctgggtcttcttggcgacac

SEQ ID NO: 13 - DNA, 81 bp, Artificial Sequence
  Description of Artificial Sequence Primer
ggccagtactccaccatcatcgccggtatggacttcgttgccagcgacaagaacaaccgcaactgccccaaaggtgtcgtt

SEQ ID NO: 14 - DNA, 81 bp, Artificial Sequence
  Description of Artificial Sequence Primer
gctctggaggcgggcagcggcgctgttcacggaggaggagtaaccaccgcccagggataaggaggcaacgacacctttggg

SEQ ID NO: 15 - DNA, 82 bp, Artificial Sequence
  Description of Artificial Sequence Primer
gcccgcctccagagctctggtgtcatggtcgccgtcgctgccggtaacaacaacgctgacgcccgcaactactcccctgctt

SEQ ID NO: 16 - DNA, 80 bp, Artificial Sequence
  Description of Artificial Sequence Primer
gttggagaagctggagcggcggtcgtagcggtcagaagcaccgacggtgcagaccgagggctcagaagcaggggagtagt

SEQ ID NO: 17 - DNA, 83 bp, Artificial Sequence
  Description of Artificial Sequence Primer
ctccagcttctccaactacggcagcgttttggacatcttcggccctggtaccagcatcctctccacctggatcggcggcagca

SEQ ID NO: 18 - DNA, 81 bp, Artificial Sequence
  Description of Artificial Sequence Primer
tcatgaggtaggcagcgagaccggcaacgtggggagtagccatggaggtaccagagatggagcgggtgctgccgccgatcc

SEQ ID NO: 19 - DNA, 81 bp, Artificial Sequence
  Description of Artificial Sequence Primer
ctgcctacctcatgaccttaggaaagaccaccgccgccagcgcttgccgttacatcgccgacaccgccaacaagggcgact

SEQ ID NO: 20 - DNA, 87 bp, Artificial Sequence
  Description of Artificial Sequence Primer
atataagcttctattaagcctggtagttgttgtaggctaacaggttgacggtgccgaagggaatgttgcttaagtcgcccttgttgg

SEQ ID NO: 21 - DNA, 36 bp, Artificial Sequence
  Description of Artificial Sequence Primer
aattcatgagaggatcgcatcagcatcagcatcagg

SEQ ID NO: 22 - DNA, 36 bp, Artificial Sequence
  Description of Artificial Sequence Primer
gatccctgatgctgatgctgatgcgatcctctcatg

SEQ ID NO: 23 - DNA, 29 bp, Artificial Sequence
  Description of Artificial Sequence Primer
gcggatccgctcctgccgttgagcagcgc

SEQ ID NO: 24 - DNA, 44 bp, Artificial Sequence
  Description of Artificial Sequence Primer
gcggatccgatgacgatgacaaagctcctgccgttgagcagcgc